(19) United States
(12) Patent Application Publication
(10) Pub. No.: US 2010/0048519 A1
(43) Pub. Date: Feb. 25, 2010

(54) METHODS OF TREATMENT FOR ULCERATIVE COLITIS USING AMINOSALICYLATE

(76) Inventors: Chyon-Hwa Yeh, Cincinnati, OH (US); Linda Mary Law, Geneva (CH); Gino Regalli, Montgomery, OH (US); Nora Lee Zorich, Cincinnati, OH (US); Joan Marie Meyer, Wyoming, OH (US); Pamela Jean Schofield, Cincinnati, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/376,168

(22) PCT Filed: Sep. 13, 2006
(86) PCT No.: PCT/US06/35428

§ 371 Date: Oct. 7, 2009

(51) Int. Cl. A61K 31/606; A61P 1/00

(52) U.S. Cl. 514/166

(57) ABSTRACT

Disclosed herein is a new treatment for ulcerative colitis in male mammalian subjects. Further disclosed are increased dosages of an aminosalicylate composition for treatment of moderate ulcerative colitis in male mammalian subjects.

### TECHNICAL FIELD

The present invention relates to new treatments of ulcerative colitis in male mammalian subjects. The invention relates to use of increased dosages of an aminosalicylate for treatment of moderate ulcerative colitis in these subjects.

### BACKGROUND OF THE INVENTION

Ulcerative colitis (UC) is a condition that causes inflammation and sores in the form of ulcers, in the lining of the rectum and colon. The inflammation may kill the cells that line the colon, causing ulcers which may then bleed and produce pus. Inflammation in the colon may also cause the colon to empty frequently, causing diarrhea. When the inflammation occurs in the rectum and lower part of the colon it is called ulcerative proctitis. If the entire colon is affected it is called pancolitis. If only the left side of the colon is affected it is called left-sided or distal colitis.

UC is a type of inflammatory bowel disease (IBD). IBD is the general name for diseases that cause inflammation in the small intestine and colon. UC is oftentimes difficult to diagnose as it shares symptoms common to other intestinal disorders and to Crohn's disease, another type of IBD. Crohn's disease differs because it causes inflammation deeper within the intestinal wall and can occur in other parts of the digestive system including the small intestine, mouth, esophagus, and stomach.

One known method of drug therapy to treat UC is administration of aminosalicylates. Aminosalicylates include 5-aminosalicylic acid (5-ASA), salts thereof, and pro-drugs that release 5-aminosalicyclic acid, or salts thereof, in vivo. Pro-drugs that release 5-aminosalicylic acid, or salts thereof, in vivo include, but are not limited to: olsalazine, balsalazide, and sulfasalazine. Aminosalicylates may be administered orally, through an enema, or in a suppository. Most people with mild or moderate ulcerative colitis are treated with aminosalicylates drugs first. The aminosalicylates also used in cases of relapse and to maintain remission.

ASACOL® is a product comprising the aminosalicylate, 5-aminosalicylic acid or mesalamine. ASACOL® is effective in treating patients with mild to moderate ulcerative colitis. Its effectiveness also extends to the maintenance of remission for prolonged periods. The current recommended dose of orally delivered ASACOL® for active disease is two 400-mg tablets three times daily for a total of 2.4 g/day (grams per day) for the treatment of mild to moderate UC. If the patient does not respond to ASACOL®, then alternatives, such as corticosteroids, are considered.

It is discovered herein that wherein the subject is a mammalian male and the UC is moderate, administration of aminosalicylate at doses in an amount to deliver greater than about 2.4 g/day, shows significant improvement in the condition.

### BRIEF SUMMARY OF THE INVENTION

In one aspect of the present invention there is method of treating moderate ulcerative colitis in a mammalian male subject comprising the step of orally administering to the mammalian male subject an aminosalicylate in an amount to deliver greater than about 2.4 g/day of 5-aminosalicylic acid to the subject. In some embodiments, the step of orally administering an aminosalicylate comprises orally administering an aminosalicylate in an amount to deliver about 4.8 g/day of 5-aminosalicylic acid to said subject. In some embodiments wherein about 4.8 g/day of 5-aminosalicylic acid is delivered to the subject, the aminosalicylate comprises mesalamine or a salt thereof. In some embodiments wherein about 4.8 g/day of 5-aminosalicylic acid is delivered to the subject, the step of orally administering comprises orally administering once per day, twice per day, three times per day, or four times per day. In some embodiments, the aminosalicylate comprises mesalamine or a salt thereof. In certain embodiments, the aminosalicylate comprises mesalamine and further wherein the mesalamine is administered in an amount of about 4.8 g/day. In certain embodiments, the mammalian male subject is a human male. In certain embodiments, the mammalian male subject is less than about 65 years of age. In certain embodiments, the mammalian male subject is a non-smoking subject. In certain embodiments, the mammalian male subject is Caucasian. In certain embodiments, the mammalian male subject is a previous or current steroid user. In some embodiments, the step of orally administering comprises orally administering tablets comprising about 800 milligrams of mesalamine or a salt thereof. In some embodiments wherein tablets comprising about 800 milligrams of mesalamine or a salt thereof are orally administered, the tablets are delayed-release tablets. In some embodiments, the step of orally administering comprises orally administering tablets comprising about 1.2 g mesalamine or a salt thereof. In some embodiments wherein tablets comprising about 1.2 g of mesalamine or a salt thereof are orally administered, the tablets are delayed-release tablets. In some embodiments, the aminosalicylate comprises a component selected from the group consisting of mesalamine, a salt of mesalamine, olsalazine, a salt of olsalazine, balsalazide, a salt of balsalazide, sulfasalazine, a salt of sulfasalazine, or any pharmaceutically acceptable combination thereof. In some embodiments, the step of orally administering comprises orally administering once per day, twice per day, three times per day, or four times per day.

In another aspect of the present invention, there is method of treating moderate ulcerative colitis in a mammalian male subject comprising the step of administering to the mammalian male subject an aminosalicylate in an amount to deliver greater than about 2.4 g/day but less than or equal to about 4.8 g/day of 5-aminosalicylic acid to the subject. In certain embodiments, the step of administering comprises rectal administration. In some embodiments, the step of administering an aminosalicylate comprises administering an aminosalicylate in an amount to deliver about 4.8 g/day of 5-aminosalicylic acid to the subject. In some embodiments wherein about 4.8 g/day of 5-aminosalicylic acid is delivered to the subject, the aminosalicylate comprises mesalamine or a salt thereof. In some embodiments wherein about 4.8 g/day of 5-aminosalicylic acid is delivered to the subject, the step of administering comprises administering once per day, twice per day, three times per day, or four times per day. In some embodiments, the aminosalicylate comprises mesalamine or a salt thereof. In some embodiments, the aminosalicylate comprises mesalamine and further wherein the mesalamine is administered in an amount of about 4.8 g/day. In certain embodiments, the mammalian male subject is a human male. In certain embodiments, the mammalian male subject is less than about 65 years of age. In certain embodiments, the mammalian male subject is a non-smoking subject. In certain embodiments, the mammalian male subject is Caucasian. In certain embodiments, the mammalian male subject is a previous or current steroid user. In certain embodiments, the step of administering comprises administering a rectal composition comprising about 800 milligrams or about 1.2 g of mesalamine or a salt thereof. In some embodiments, the rectal composition is an enema. In some embodiments, the rectal composition is a foamed composition. In some embodiments wherein a rectal composition is administered, the rectal composition is a suppository. In some embodiments, the aminosalicylate comprises a component selected from the group consisting of mesalamine, a salt of mesalamine, olsalazine, a salt of olsalazine, balsalazide, a salt of balsalazide, sulfasalazine, a salt of sulfasalazine, or any pharmaceutically acceptable combination thereof. In some embodiments, the step of administering comprises administering once per day, twice per day, three times per day, or four times per day.

The foregoing has outlined the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 (a) is a subgroup analysis of treatment outcome at week 6 for male patients with moderate UC of data from two phase III, multi-center, randomized, double-blind, controlled clinical trials of identical design (Study 1 and Study 2) combined; (b) is a subgroup analysis of treatment outcome at week 6 for female patients with moderate UC for Study 1 and Study 2 combined.

FIG. 2 (a) is a subgroup analysis of treatment outcome at week 6 for male patients with moderate UC for Study 1 and Study 2 combined; (b) is a subgroup analysis of treatment outcome at week 6 for female patients with moderate UC for Study 1 and Study 2 combined.

FIG. 3 (a) is a subgroup analysis of treatment outcome at week 6 for male patients with moderate UC for Study 1 and Study 2 UC; (b) is a subgroup analysis of treatment outcome at week 6 for female patients with moderate UC for Study 1 and Study 2 combined.

### DETAILED DESCRIPTION OF THE INVENTION

As used herein, “a” or “an” means one or more. Unless otherwise indicated, the singular contains the plural and the plural contains the singular.

As used herein, “aminosalicylate” refers to a class of compounds capable of releasing 5-amino-2-hydroxybenzoate or 5-amino-2-hydroxybenzoic acid as an active moiety in vivo. Non-limiting examples include mesalamine (5-amino-2-hydroxybenzoic acid), olsalazine (3,3′-dicarboxy-4,4′-dihydroxyazobenzene), balsalazide ((E)-5-[[4-[[(2-carboxyethyl)amino]carbonyl]phenyl]azo]-2-hydroxybenzoic acid), and sulfasalazine (2-hydroxy-5-[[4-[(2-pyridinylamino)sulfonyl]phenyl]azo]-benzoic acid). Although the examples provided describe the free acid, free amine forms, the term is not so limited and encompasses the free acid forms, the free amine forms, and any salts thereof. A composition comprising an aminosalicylate may have one or greater than one aminosalicylate in addition to other possible components. The active moiety is illustrated below:

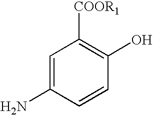

wherein R₁ can be hydrogen or a physiologically relevant counterion and the nitrogen can be further protonated and carry a positive charge along with a physiologically relevant counterion.

As used herein, “mesalamine” means 5-amino-2-hydroxybenzoic acid. The term “mesalamine” covers the free acid, the free amine, and any salts thereof. The term “mesalamine” may also be used interchangeably with “mesalazine”, “5-ASA” or “5-aminosalicylic acid”.

As used herein, the term “moderate” in relationship to ulcerative colitis will be commonly understood in the art and means a level of UC disease activity in which the subject exhibits rectal bleeding and colon wall friability, with an absence of, or insignificant, systemic toxicity. Determination of moderate UC will therefore be consistent with Kombluth et al., “Ulcerative colitis practice guidelines in adults (update) ACG”, Practice parameters committee, Am. J. Gastroenterol. 2004, 99: 1371-1385.

As used herein, the term “non-smoking subject” means a subject that does not smoke cigarettes, cigars, or the like concurrent with practice of the method herein.

As used herein, the term “previous or current steroid-using” with reference to the mammalian (e.g., human) subject under treatment means that the subject currently (i.e., concurrent with practice of the method herein) or previously has used (i.e., prior to practice of the method herein) a steroid therapy to treat ulcerative colitis.

As used herein, “treating” refers to the amelioration and/or delay of at least one symptom of a medical condition and in particular embodiments does not necessarily encompass a cure for the medical condition.

The inventors find herein that administration of an amount of aminosalicylate to deliver greater than about 2.4 g/day and in certain embodiments less than or equal to about 4.8 g/day of 5-aminosalicylic acid (5-ASA) to a mammalian male subject with moderate Ulcerative colitis provides superior therapeutic benefits in comparison to the delivery of 2.4 g/day of 5-ASA that is typically provided to such patients. In certain embodiments, the route of administration is oral administration in the form of tablets, in certain embodiments delayed-release tablets. However, other forms of administration, particularly rectal administration, also benefit from the new regimen and is therefore within the scope of the present invention. Where rectal administration is used, enemas or foamed compositions are the preferred dosage form.

The amount of 5-ASA administered is determined using the ratio of molecular weights of the aminosalicylate and the molecular weight of 5-ASA along with the number of moles of 5-ASA delivered by that aminosalicylate. When the aminosalicylate is mesalamine, the molecular weight ratio is unity and the amount administered is equal to the weight of 5-ASA delivered.

### Example 1

Administration of mesalamine at 4.8 g/day via three daily doses consisting of two, 800 mg dosage tablets provides a clear efficacy benefit over 2.4 g/day of mesalamine given as three daily doses consisting of two, 400 mg dosage tablets and addresses an unmet medical need for men with moderate ulcerative colitis. The safety profile of the 4.8 g/day regimen is comparable to that of the 2.4 g/day regimen.

A non-limiting suitable example of an 800 mg dosage and other dosage forms are described in U.S. Pat. No. 6,893,662 issued to Dittmar et al. on May 17, 2005. Suitable, non-limiting examples of 400 mg and other dosage forms may be found in U.S. Pat. No. 5,541,170 issued to Rhodes, et al. on Jul. 30, 1996. Additionally, suitable, non-limiting examples of 1.2 g and other dosage forms may be found in U.S. Pat. No. 6,773,720 issued to Villa, et al. on Aug. 10, 2004.

Data from patients with mild to moderate Ulcerative colitis (UC) are combined and analyzed from two phase III, multi-center, randomized, double-blind, controlled clinical trials of identical design assessing the safety and clinical efficacy of the increased dosage of 5-ASA. The two clinical trials are referred to herein as “Study 1” and “Study 2.”

The primary endpoint is the percentage of moderate UC patients achieving overall improvement (i.e., treatment success) from baseline at week 6. This is defined as: (1) complete response (remission); complete resolution of signs and symptoms (stool frequency, rectal bleeding, Patient Functional Assessment (PFA) and sigmoidoscopy score) and a Physician's Global Assessment (PGA) of 0; or (2) partial response; improvement from baseline in the PGA score and improvement in at least one clinical assessment (stool frequency, rectal bleeding, PFA or sigmoidoscopy score) and no worsening in any of the other clinical assessments.

Results of the primary analysis in patients with moderate disease remain statistically significant after adjustment for demographic or baseline characteristics using the Cochram-Mantel-Haenszel test stratified by subgroup variable.

Pre-specified subgroup analyses for fifty-four demographic and baseline characteristics are performed in patients with moderately active disease (PGA score=2) to assess consistency of primary endpoint.

Analysis of the efficacy data in men with moderate disease demonstrates significant benefit from the 4.8 g/day regimen compared to the lower dose in this population in both studies, whether analyzed according to the pre-specified primary analysis or using set-to-failure (Table 1). The robustness of the results in men is supported by the consistency of the results for the primary analysis and for the set-to-failure analyses as shown in Table 1.

TABLE 1
Success Rates in Male Human Patients with Moderate Disease
at Baseline.

| | | 2.4 g/ | | 4.8 g/ | Difference in | |
|---|---|---|---|---|---|---|
| | n | day | n | day | Proportions | p-value |
| Primary | | | | | | |
| Analysis | | | | | | |
| Study 1 | 58 | 50.0% | 53 | 75.5% | 25.5% | 0.0057 |
| Study 2 | 43 | 48.8% | 38 | 76.3% | 27.5% | 0.0111 |
| Set-to-Failure | | | | | | |
| Study 1 | 62 | 46.84% | 54 | 74.1% | 27.3% | 0.0028 |
| Study 2 | 44 | 47.7% | 40 | 72.5% | 24.8% | 0.0209 |

The results in men with moderate disease are consistent with the expected success rates used to design both studies. In designing these studies, the sample size is based on the following assumptions: the success rate for the 2.4 g/day treatment group would be 40% and the success rate for the 4.8 g/day treatment group would be 60%. Thus, the hypothesized true difference between treatment groups is 20%. Observed differences of approximately 25% in men are consistent with the hypothesized value.

The results show consistency across the multiple pre-specified subpopulation analyses within each study regardless of the analyses (i.e., set to failure) performed. To further evaluate the robustness of the results in men with moderate disease, whisker plots (i.e., point estimate and 95% confidence interval for the difference between the 2.4 g/day group and the 4.8 g/day group) for various subgroups defined on the basis of baseline characteristics (e.g., disease severity, demographic parameters) are prepared.

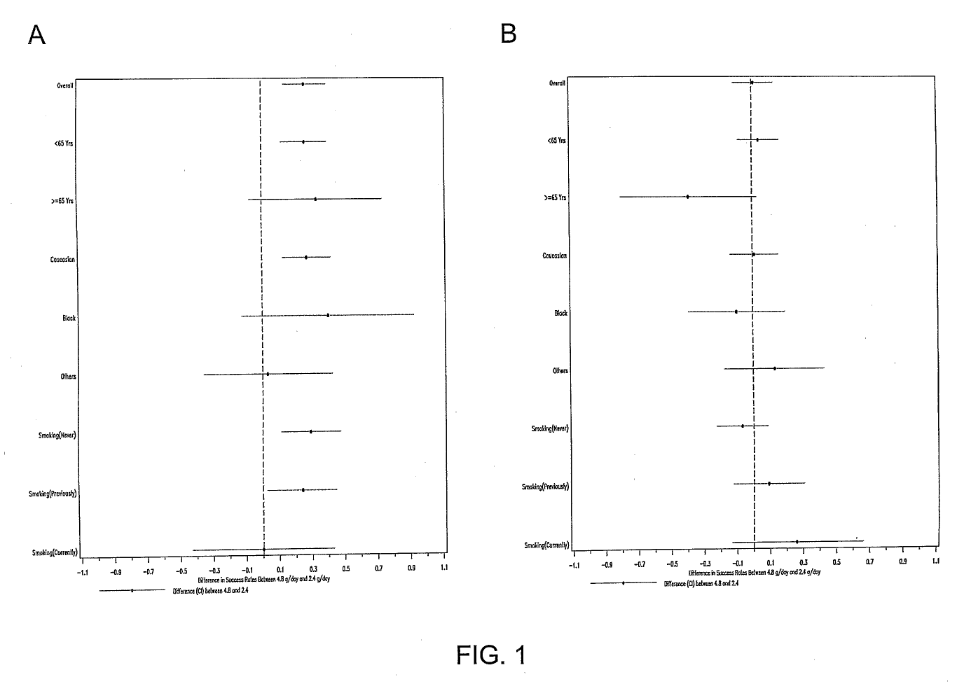

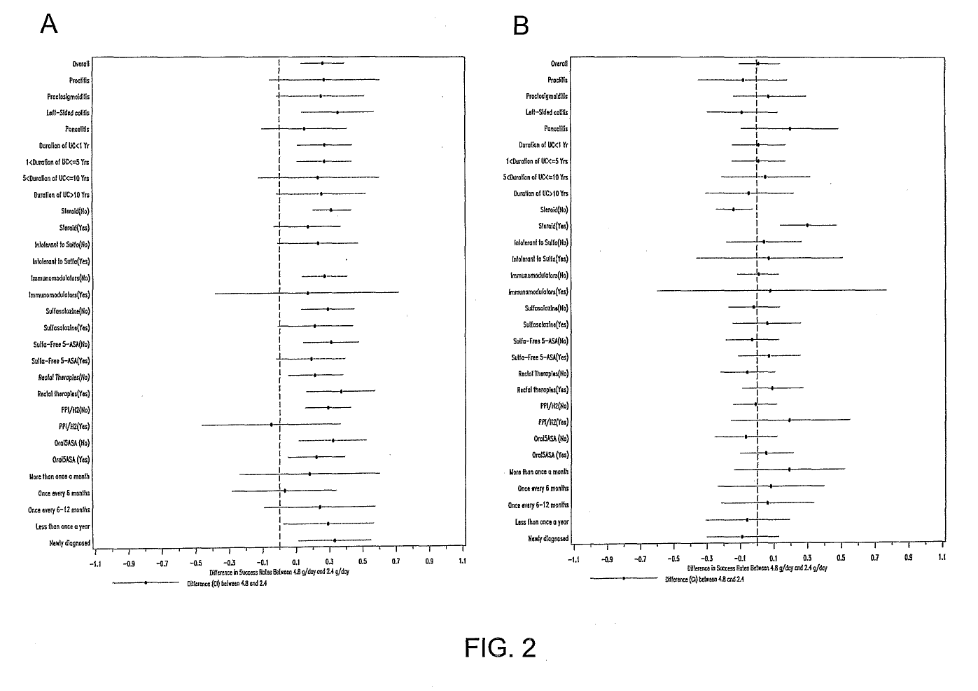

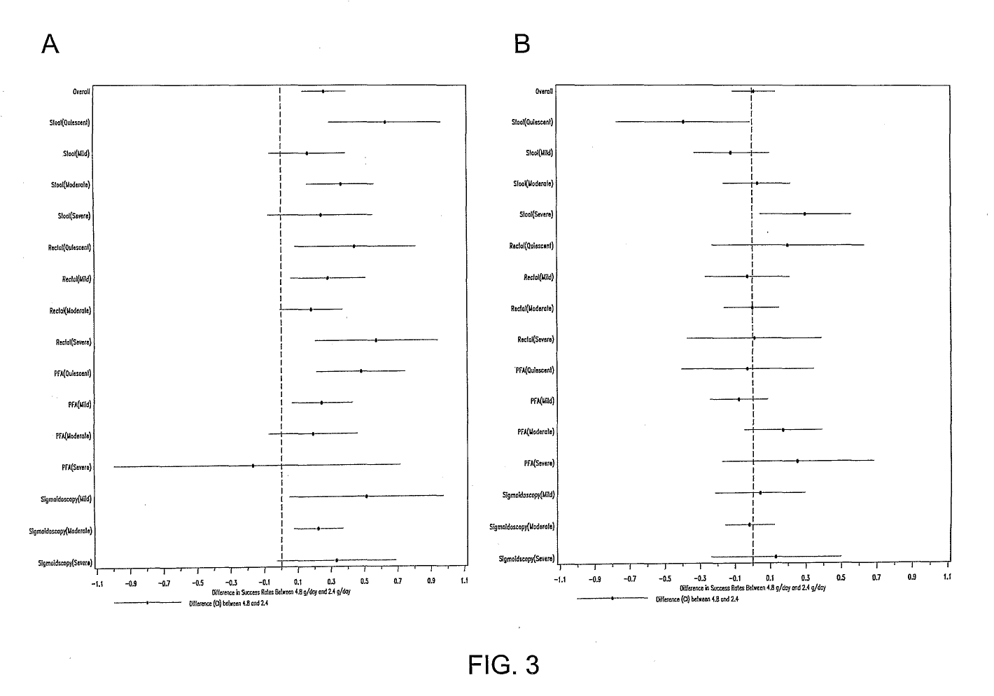

FIGS. 1-3 demonstrate the difference between treatment groups in response rates with 95% confidence intervals for a variety of subpopulations that are pre-specified in the statistical analysis plans. FIG. 1 (a) is a subgroup analysis of treatment outcome at week 6 for male patients with moderate disease for Study 1 and Study 2 combined; (b) is a subgroup analysis of treatment outcome at week 6 for female patients with moderate disease for Study 1 and Study 2 combined. FIG. 2 (a) is a subgroup analysis of treatment outcome at week 6 for male patients with moderate disease for Study 1 and Study 2 combined; (b) is a subgroup analysis of treatment outcome at week 6 for female patients with moderate disease for Study 1 and Study 2 combined. FIG. 3 (a) is a subgroup analysis of treatment outcome at week 6 for male patients with moderate disease for Study 1 and Study 2 combined; (b) is a subgroup analysis of treatment outcome at week 6 for female patients with moderate disease for Study 1 and Study 2 combined. The results are defined on the basis of the following characteristics:
Age (<65 years, ≧65 years)
Race (Caucasian, Black, Other)
Smoking (never, previously, currently)
Disease location (proctitus, left-sided colitis, pancolitis)
Duration of ulcerative colitis (<1 year, >1 year and ≧5 years, >5 years and ≦10 years, >10 years)
Drug history
   Use of steroids (yes/no)
   Intolerant to sulfa (yes/no)
   Use of immunomodulators (yes/no)
   Use of sulfasalazine (yes/no)
   Use of sulfa-free 5-aminosalicylate (yes/no)
   Use of rectal therapies (yes/no)
   Use of PPI/H2 (yes/no)
   Use of oral 5-ASA (yes/no)

Frequency of flares (>1 per month, 1 per 6 months, 1 per 6-12 months, <1 per year, newly diagnosed)

Each of these subgroups is pre-specified in the statistical analysis plan prior to unblinding the study. As can be seen from the point estimates and confidence intervals for the differences between the 4.8 g/day group and the 2.4 g/day group, the 4.8 g/day group is consistently superior to the 2.4 g/day group, with the differences (51 of 54 subgroups for males with moderate disease in the combined population) being significantly favorable to the 4.8 g/day group for non-smokers, Caucasians, previous or current steroid users, and males less than about 65 years of age.

In addition, a Bayesian analysis is conducted to calculate the probability that treatment with the 4.8 g/day dose in men results in a higher success rate than treatment with the 2.4 g/day dose. This type of analysis considers data from any previous studies conducted using either of the two dose levels. Success rates from male patients with moderate disease at baseline from three previous mesalamine studies are used to estimate the prior distributions for the 2.4 g/day dose and the 4.8 g/day dose. Using these prior distributions and Bayes analyses, the “posterior distribution” is calculated for each dose level based on the data obtained from previous studies.

The results from the Bayes analysis show that the probability of a successful treatment outcome using the 4.8 g/day dose is 74.7% (95% credible interval: 64.9%, 83.5%). The interpretation of the 95% credible interval means that there is a 95% probability that the success rate for the 4.8 g/day dose is between 64.9% and 83.5%.

The probability of a successful treatment outcome in men using the 2.4 g/day dose is 47.2% (95% credible interval: 35.7%, 58.9%). There is a 95% probability that the success rate for the 2.4 g/day dose is between 35.7% and 58.9%. The probability that treatment with 4.8 g/day dose in men will result in a higher success rate than the 2.4 g/day dose is 99.97%. The analysis further supports the robustness of the results from the male population in the clinical program.

### Conclusions

Among male patients with moderate disease, overall improvement with a 4.8 g/day delayed-release oral mesalamine (800 mg tablet) is consistent across a wide variety of patient subgroups. The incremental benefit of a 4.8 g/day over 2.4 g/day is more apparent in patients previously treated with steroids. Benefits of the 4.8 g/day regimen are also apparent in Caucasians, non-smokers, and males less than 65 years of age. No baseline or demographic characteristics predict treatment failure. Both 4.8 g/day delayed-release oral mesalamine (800 mg tablet) and 2.4 g/day (400 mg tablet) are well tolerated and had similar safety profiles.

### Example 2

A 70 kg man diagnosed with moderate Ulcerative colitis is prescribed a pharmaceutical oral composition comprising 1.2 g of olsalazine (mol. wt. 302.24), a mesalamine dimer, two tablets to be taken twice daily for a total of 4.8 g/day of the 5-ASA dimer (5-ASA mol. wt.=153.14). The patient takes two tablets of the pharmaceutical in the morning and two tablets in the evening such that about 4.8 g/day of 5-ASA is delivered. The Physician's Global Assessment (PGA) score improves in comparison to baseline and rectal bleeding reduces.

Molecular weights and the moles of 5-ASA delivered per mole of aminosalicylate are used to determine the amount of 5-ASA delivered when the aminosalicylate to be administered is one other than mesalamine. Complete cleavage of pro-drug forms is assumed. For example, for the aminosalicylate olsalazine (OLSAL), the following equation is used to determine the approximate weight of olsalazine needed to deliver a targeted amount of about 4.8 g of 5-aminosalicylic acid:

(4.8 g 5-ASA)*(1 mole 5-ASA/153.14 g 5-ASA)*(1 mole OLSAL/2 mole 5-ASA)*(302.24 g OLSAL/1 mole OLSAL)=4.7 g OLSAL; wherein * signifies multiplication.

### Using a Twice Daily Regimen:

(4.7 g OLSAL/2 times per day)=2.4 g OLSAL at each time per day, which can be administered in the form of two tablets containing 1.2 g of OLSAL in the morning and evening.

Molecular weights and in the number of moles of 5-ASA delivered per mole of aminosalicylate are shown in Table 2 below for some illustrative aminosalicylates.

TABLE 2
Molecular Weights and Moles of 5-ASA Delivered for Some
Aminosalicylates.

| | Moles of 5-ASA per | |
|---|---|---|
| Aminosalicylate (as free | mole of | |
| acid) | Aminosalicylate | Molecular weight |
| Mesalamine | 1 | 153.14 |
| Balsalazide | 1 | 357.32 |
| Olsalazine | 2 | 302.24 |
| Sulfasalazine | 1 | 398.40 |

### Example 3

A 72 kg man diagnosed with moderate Ulcerative colitis is prescribed a pharmaceutical composition comprising four 1.2 g mesalamine delayed-release tablets, to be taken once daily. The four tablets are taken in the morning so that 4.8 g/day of 5-ASA is delivered. The Physician's Global Assessment (PGA) score improves in comparison to baseline and rectal bleeding reduces.

### Example 4

A 75 kg man diagnosed with moderate Ulcerative colitis is prescribed a pharmaceutical oral composition comprising two 1.2 g balsalazide delayed-release tablets, to be taken three times daily for a total of 7.2 g/day of balsalazide; this regimen is calculated to deliver about 3.1 g of 5-ASA. The Physician's Global Assessment (PGA) score improves in comparison to baseline and rectal bleeding reduces.

### Example 5

A 71 kg man diagnosed with moderate Ulcerative colitis is prescribed a pharmaceutical composition comprising a rectal mesalamine foam. The foam is administered three times per day (morning, afternoon, and evening) such that 1 g of mesalamine is administered at each interval for a total of 3 g of mesalamine per day. The Physician's Global Assessment (PGA) score improves in comparison to baseline and rectal bleeding is reduced.

Example 5 provides an embodiment of the present invention using non-oral administration of aminosalicylate. Non-limiting suitable examples of a rectal composition are described in U.S. Pat. No. 5,082,651 issued to Healey et al. on Jan. 21, 1992.

Aminosalicylate treatment may be used to deliver weights of 5-ASA which are greater than the prior art 2.4 g/day, up to and including a daily dosage of about 4.8 g/day. This range includes delivered dosages of 2.5 g/day, 2.6 g/day, 2.7 g/day, 2.8 g/day, 2.9 g/day, 3.0 g/day, 3.1 g/day, 3.2 g/day, 3.3 g/day, 3.4 g/day, 3.5 g/day, 3.6 g/day, 3.7 g/day, 3.8 g/day, 3.9 g/day, 4.0 g/day, 4.1 g/day, 4.2 g/day, 4.3 g/day, 4.4 g/day, 4.5 g/day, 4.6 g/day, 4.7 g/day, and 4.8 g/day of aminosalicylate (e.g., 5-ASA), as well as numerical values in-between the stated dosages. In certain embodiments the aminosalicylate is 5-ASA. In certain embodiments, the delivered dosage is 4.8 g/day of 5-ASA.

While the certain administration comprises administration of three daily doses, the administration may comprise other schedules, such as, but not limited to, administration once per day, administration twice per day, administration three times per day, and administration four times per day.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, manufacture, composition of matter, means, methods and steps described in the specification. As one of ordinary skill in the art will readily appreciate from the disclosure of the present invention, processes, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, manufacture, compositions of matter, means, methods, or steps.

What is claimed is:

1. A method of treating moderate ulcerative colitis in a mammalian male subject comprising the step of administering to said mammalian male subject an aminosalicylate in an amount to deliver greater than about 2.4 g/day of 5-aminosalicylic acid to said subject.

2. The method of claim 1, wherein the mammalian male subject is a human male.

3. The method of claim 2, comprising the step of orally administering to said mammalian male subject an aminosalicylate in an amount to deliver greater than about 2.4 g/day but less than or equal to about 4.8 g/day of 5-aminosalicylic acid to said subject.

4. The method of claim 3, wherein said step of orally administering an aminosalicylate comprises orally administering an aminosalicylate in an amount to deliver about 4.8 g/day of 5-aminosalicylic acid to said subject.

5. The method of claim 4, wherein said aminosalicylate comprises mesalamine or a salt thereof.

6. The method of claim 5, wherein said step of orally administering comprises orally administering once per day, twice per day, three times per day, or four times per day.

7. The method of claim 3, wherein said aminosalicylate comprises mesalamine or a salt thereof.

8. The method of claim 3, wherein said aminosalicylate comprises mesalamine and further wherein said mesalamine is administered in an amount of about 4.8 g/day.

9. The method of claim 3, wherein said mammalian male subject is less than 65 years of age.

10. The method of claim 3, wherein said mammalian male subject is a non-smoking subject.

11. The method of claim 3, wherein said mammalian male subject is Caucasian.

12. The method of claim 3, wherein said mammalian male subject is a previous or current steroid user.

13. The method of claim 3, wherein said step of orally administering comprises orally administering a tablet comprising about 800 milligrams of mesalamine or a salt thereof.

14. The method of claim 13, wherein said tablet is a delayed-release tablet.

15. The method of claim 3, wherein said aminosalicylate comprises a component selected from the group consisting of mesalamine, salts of mesalamine, olsalazine, salts of olsalazine, balsalazide, salts of balsalazide, sulfasalazine, salts of sulfasalazine, and mixtures thereof.

16. The method of claim 3, wherein said step of orally administering comprises orally administering once per day, twice per day, three times per day, or four times per day.

17. The method of claim 2, wherein step of administering comprises rectal administration.

18. The method of claim 17, wherein said step of administering an aminosalicylate comprises administering an aminosalicylate in an amount to deliver about 4.8 g/day of 5-aminosalicylic acid to said subject.

19. The method of claim 18, wherein said aminosalicylate comprises mesalamine or a salt thereof.

20. The method of claim 19, wherein said step of administering comprises administering once per day, twice per day, three times per day, or four times per day.

21. The method of claim 17, wherein said aminosalicylate comprises mesalamine or a salt thereof.

22. The method of claim 17, wherein said mammalian male subject is less than 65 years of age.

23. The method of claim 17, wherein said mammalian male subject is a non-smoking subject.

24. The method of claim 17, wherein said mammalian male subject is Caucasian.

25. The method of claim 17, wherein said mammalian male subject is a previous or current steroid user.

26. The method of claim 17, wherein said step of administering comprises administering a rectal composition comprising about 800 milligrams or about 1.2 g of mesalamine or a salt thereof.

27. The method of claim 17, wherein said rectal composition is an enema.

28. The method of claim 17, wherein said rectal composition is a foam.

29. The method of claim 17, wherein said aminosalicylate comprises a component selected from the group consisting of mesalamine, a salt of mesalamine, olsalazine, a salt of olsalazine, balsalazide, a salt of balsalazide, sulfasalazine, a salt of sulfasalazine, or any pharmaceutically acceptable combination thereof.

30. The method of claim 29, wherein said step of administering comprises administering once per day, twice per day, three times per day, or four times per day.
